(19) United States
(12) Patent Application Publication
(10) Pub. No.: US 2004/0143322 A1
(43) Pub. Date: Jul. 22, 2004

(54) Method and apparatus for treating vulnerable artherosclerotic plaque

(75) Inventors: Frank Litvack, Los Angeles, CA (US); Theodore L. Parker, Danville, CA (US)

Correspondence Address: BURNS DOANE SWECKER & MATHIS L L P, POST OFFICE BOX 1404, ALEXANDRIA, VA, 22313-1404, US

(73) Assignee: Conor Medsystems, Inc.

(21) Appl. No.: 10/705,424

(22) Filed: Nov. 10, 2003

Related U.S. Application Data
(60) Provisional application No. 60/425,096, filed on Nov. 8, 2002.

(51) Int. Cl. A61F 2/06

(52) U.S. Cl. 623/1.42

(57) ABSTRACT

Methods and apparatus for treatment of vulnerable plaque provide local delivery of one or more plaque stabilizing agents. Delivery of the plaque stabilizing agents described herein stabilize vulnerable plaques at and downstream of an implantation site can reduce the occurrence of rupture of these plaques. An expandable medical device for delivering a therapeutic agent locally to a vulnerable plaque includes an implantable medical device body configured to be implanted within a coronary artery, and a therapeutic dosage of a therapeutic agent for stabilization of vulnerable plaque. The therapeutic agent is affixed in openings in the implantable medical device body in a manner such that the therapeutic agent is released to the vulnerable plaque at a therapeutic dosage and over an administration period effective to stabilize the vulnerable plaque.

### CROSS REFERENCE TO RELATED APPLICATIONS

This application claims priority to U.S. Provisional Application Serial No. 60/425,096 filed Nov. 8, 2002, which is incorporated herein by reference in its entirety.

### BACKGROUND

Heart disease is the leading cause of death for both men and women in the world today. It is characterized by deposits of fat, fibrin, cellular debris, and calcium on or within the arterial walls. Atherosclerotic plaque which develops in the vessels can partially or fully occlude the coronary arteries. When these coronary arteries become blocked, symptoms ranging from angina to heart attacks, may occur. In a percentage of these cases, the coronary arteries may be unblocked through a non-invasive technique such as balloon angioplasty. In other cases a bypass of the occluded or blocked vessel may be necessary.

In coronary artery disease, the fatal heart attacks are often caused by sudden blockages that are created, not by the slow accumulation of plaque that gradually block off the arteries, but by a sudden thrombosis (clotting) of the arteries caused by what are now referred to as “vulnerable plaque.” Vulnerable plaques are defined as plaques prone, in the presence of an appropriate trigger, to events such as ulceration rupture, erosion, or thrombus. It has been found that the rupture-prone (i.e., vulnerable plaques) typically have a thin fibrous cap, numerous inflammatory cells, a substantial lipid core, and few smooth muscle cells. Many of these so-called “vulnerable plaques” do not block the arteries and do not limit the blood flow through the blood vessels. On the other hand, much like an abscess, they are ingrained in the arterial wall, so that they are undetectable by traditional methods. It has recently been appreciated that vulnerable plaques which do not limit flow may be particularly dangerous because they can go undetected and then rupture suddenly causing heart attack and death. For a variety of reasons, the vulnerable plaques are more likely to erode or rupture, creating thrombosis and a raw tissue surface that forms scabs. Thus, they may be more dangerous than other plaques that cause pain, and may be responsible for as much as 60-80% of all heart attacks.

Traditional methods of diagnosing arterial disease, such as stress tests and angiograms, are inadequate at detecting these vulnerable plaques. They cannot be seen by conventional angiography or fluoroscopy. Therefore, in many instances, this potentially lethal condition goes untreated.

At present, methods are being developed which allow a physician to view vulnerable plaque. Several invasive and non-invasive imaging techniques are available to assess atherosclerotic disease vessels. For example, it has been observed that the inflamed necrotic core of a vulnerable plaque maintains itself at a temperature which may be one or more degrees Celsius higher than the surrounding tissue. Thermal sensors that measure the temperature of the arterial wall on the premise that the inflammatory process at the root of vulnerable plaque generates heat have been used to map vulnerable plaques. Other new technologies under development include magnetic resonance imaging (MRI), elastography used to identify different plaque components with intravascular ultrasound by analyzing possible differences in the elastic features of multiple plaque structures, optical coherence tomography (OCT), contrast agents, near-infrared and infrared light techniques, or accumulation of radiopharmaceutical agents. These techniques will improve the ability to identify the composition of the atherosclerotic plaque in the vessel wall and may be capable of conclusively identifying the vulnerable plaques.

Compounds capable of stabilizing vulnerable plaques represent important therapeutic agents. However, the delivery of stabilizing compounds is limited by the high dosages needed, unsuitability for systemic delivery, and inability to get the appropriate dosages delivered over extended administration periods when needed.

### SUMMARY OF THE INVENTION

The present invention relates to the local delivery of therapeutic agents which stabilize vulnerable plaque. The therapeutic agents are delivered by a stent locally to the blood vessel walls over an administration period sufficient to achieve stabilization of the vulnerable plaque.

In accordance with one aspect of the present invention, a method for treating vulnerable plaque within a blood vessel includes the steps of identifying an implantation site in a blood vessel with vulnerable plaque, wherein the implantation site is at or upstream of the vulnerable plaque, delivering an expandable medical device containing a therapeutic agent which stabilizes the vulnerable plaque to the blood vessel at the selected implantation site, implanting the medical device at the implantation site, and delivering the therapeutic agent from the expandable medical device to vessel wall tissue over an administration period sufficient to stabilize the vulnerable plaque.

In accordance with another aspect of the present invention, an expandable medical device for delivering a therapeutic agent locally to a vulnerable plaque includes an implantable medical device body configured to be implanted within a coronary artery; and a therapeutic dosage of a therapeutic agent for stabilization of vulnerable plaque, the therapeutic agent affixed in openings in the implantable medical device body in a manner such that the therapeutic agent is released to the vulnerable plaque at a therapeutic dosage and over an administration period effective to stabilize the vulnerable plaque.

### BRIEF DESCRIPTION OF THE DRAWINGS

The invention will now be described in greater detail with reference to the preferred embodiments illustrated in the accompanying drawings, in which like elements bear like reference numerals, and wherein:

FIG. 1 is a cross-sectional perspective view of a portion of an expandable medical device implanted in the lumen of an artery with a therapeutic agent arranged for delivery to the walls of the artery;

FIG. 2 is a perspective view of an expandable medical device showing a plurality of openings;

FIG. 3 is an expanded side view of a portion of the expandable medical device of FIG. 2;

FIG. 4 is an enlarged cross-section of an opening illustrating a therapeutic agent for delivery to the walls of a blood vessel;

FIG. 5 is an enlarged cross-section of an opening illustrating a first therapeutic agent and a second therapeutic agent in layers; and

FIG. 6 is an enlarged cross-section of an opening illustrating first and second therapeutic agents in concentration gradients in a matrix.

### DETAILED DESCRIPTION

The present invention relates to methods and apparatus for treatment of vulnerable plaque by local delivery of one or more plaque stabilizing agents. Vulnerable plaques can rupture creating emboli and raw tissue surfaces that can lead to thrombosis resulting in acute myocardial infarction or stroke. Delivery of the agents described herein which stabilize vulnerable plaques by a local delivery device in the form of a drug delivery stent can reduce the occurrence of rupture of these plaques.

First, the following terms, as used herein, shall have the following meanings:

The terms “drug” and “therapeutic agent” are used interchangeably to refer to any therapeutically active substance that is delivered to a bodily conduit of a living being to produce a desired, usually beneficial, effect.

The term “matrix” or “biocompatible matrix” are used interchangeably to refer to a medium or material that, upon implantation in a subject, does not elicit a detrimental response sufficient to result in the rejection of the matrix. The matrix typically does not provide any therapeutic responses itself, though the matrix may contain or surround a therapeutic agent, and/or modulate the release of the therapeutic agent into the body. A matrix is also a medium that may simply provide support, structural integrity or structural barriers. The matrix may be polymeric, non-polymeric, hydrophobic, hydrophilic, lipophilic, amphiphilic, and the like. The matrix may be bioresorbable or non-bioresorbable.

The term “bioresorbable” refers to a matrix, as defined herein, that can be broken down by either chemical or physical process, upon interaction with a physiological environment. The matrix can erode or dissolve. A bioresorbable matrix serves a temporary function in the body, such as drug delivery, and is then degraded or broken into components that are metabolizable or excretable, over a period of time from minutes to years, preferably less than one year, while maintaining any requisite structural integrity in that same time period.

The term “openings” includes both through openings and recesses.

The term “pharmaceutically acceptable” refers to the characteristic of being non-toxic to a host or patient and suitable for maintaining the stability of a beneficial agent and allowing the delivery of the beneficial agent to target cells or tissue.

The term “polymer” refers to molecules formed from the chemical union of two or more repeating units, called monomers. Accordingly, included within the term “polymer” may be, for example, dimers, trimers and oligomers. The polymer may be synthetic, naturally-occurring or semisynthetic. In preferred form, the term “polymer” refers to molecules which typically have a M_{W} greater than about 3000 and preferably greater than about 10,000 and a M_{W} that is less than about 10 million, preferably less than about a million and more preferably less than about 200,000. Examples of polymers include but are not limited to, poly-α-hydroxy acid esters such as, polylactic acid (PLLA or DLPLA), polyglycolic acid, polylactic-co-glycolic acid (PLGA), polylactic acid-co-caprolactone; poly (block-ethylene oxide-block-lactide-co-glycolide) polymers (PEO-block-PLGA and PEO-block-PLGA-block-PEO); polyethylene glycol and polyethylene oxide, poly (block-ethylene oxide-block-propylene oxide-block-ethylene oxide); polyvinyl pyrrolidone; polyorthoesters; polysaccharides and polysaccharide derivatives such as polyhyaluronic acid, poly (glucose), polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins, such as beta-cyclo dextrin sulfo butyl ethers; polypeptides, and proteins such as polylysine, polyglutamic acid, albumin; polyanhydrides; polyhydroxy alkonoates such as polyhydroxy valerate, polyhydroxy butyrate, and the like.

The term “primarily” with respect to directional delivery, refers to an amount greater than about 50% of the total amount of beneficial agent provided to a blood vessel.

The term “restenosis” refers to the renarrowing of an artery following an angioplasty procedure which may include stenosis following stent implantation.

Methods for Locally Delivering Drugs to Stabilize Vulnerable Plaque

Implantable medical devices in the form of stents when implanted directly at a site of a vulnerable plaque can be used to deliver therapeutic agents directly to the blood vessel walls at the implantation site. These devices can also be used to deliver therapeutic agents into the blood stream for delivery to the walls of the blood vessels downstream of the implantation site. The delivery of the agent locally at the vulnerable plaque site can stabilize the plaque reducing the occurrences of ruptures and healing the raw exposed tissues from a previous rupture. The delivery of the agent downstream of the implantation site can stabilize vulnerable plaques in the downstream vessels reducing the occurance of plaque ruptures. A drug delivery stent for delivery of a therapeutic agent for treatment of vulnerable plaque can be implanted at an implantation site at the location of a vulnerable plaque in the traditional manner after angioplasty or another procedure. The drug delivery stent can also be implanted at a site upsteam of one or more vulnerable plaques to deliver plaque stabilizing agents to the vulnerable plaque(s).

The metabolic mechanisms of vulnerable plaque are not completely clear. Vulnerable plaques include a fibrous cap and a lipid core. Researchers now believe that vulnerable plaques begin by excess low density lipoprotein (LDL) particles (fat particles) accumulating in the artery wall and undergoing oxidation. The altered LDLs then stimulate an inflammatory response. The altered LDLs stimulate endothelial cells to display adhesion molecules, which latch onto monocytes and T cells in the blood and bring them into the intima. Once inside the intima, the monocytes mature into active macrophages which devour the LDLs. The macrophages together with the T cells and inflammatory molecules form the lipid core. Meanwhile smooth muscle cells of the media migrate to the top of the intima, multiple, and produce a tough fibrous matrix. The fibrous cap can be weakened by the inflammatory substances in the lipid core leading to plaque rupture.

When this inflammation is combined with other stresses, like high blood pressure, it can cause the thin covering over the plaque to rupture, crack, and bleed, spilling the lipid contents of the vulnerable plaque into the bloodstream. The sticky cytokines on the artery wall capture blood cells (mainly platelets) that rush to the site of injury. When these cells clump together, they can form a clot large enough to block the artery.

Plaques having thinner fibrous caps with lower collagen contents in the cap in combination with high lipid content in the plaque core are particularly vulnerable to rupture. As the cap thins and the lipid core increases vulnerability to rupture increases. Inflammation and infection increase plaque instability. Macrophages, T lymphocytes, mast cells, and neutrophils secrete cytokine and protolytic enzymes which contribute to plaque instability, such as by degrading the cap thickness and increasing the core size.

Vulnerable plaques may be stabilized by deployment of a stent at the plaque site. However, the stabilized plaque can be further stabilized by delivery of the stabilizing agents discussed below. Commonly multiple vulnerable plaques will be found within the coronary arteries. One or more vulnerable plaques can be stabilized by delivery of a plaque stabilizing agent from a stent to the lumen of an artery upstream of the suspected plaque sites to deliver the agent to the downstream vulnerable plaques.

Stabilization of vulnerable plaques may be achieved by toughening the plaque fibrous cap, such as by increasing smooth muscle cells. Vulnerable plaque stabilization may be achieved or development of vulnerable plaques may be decreased by increasing the rate at which cholesterol is removed from the blood vessel walls by local delivery of high density lipoprotein (HDL).

Anti-inflammatory drugs that dampen the inflammatory response delivered locally at a vulnerable plaque site may stabilize the vulnerable plaque. Stabilization may also be achieved by inhibiting thrombin, preventing thrombi generation, blocking the initiation of coagulation, inhibiting platelet activation, and increasing fibrinolysis. Anti-lymphocytes, anti-macrophage substances, cyclooxygenase inhibitors, anti-metabolites, P par agonists, anti-oxidants, cholesterol-lowering drugs, antithrombotics, statins and angiotens in converting enzyme (ACE), fibrinolytics, inhibitors or the intrinsic coagulation cascade, antihyperlipoproteinemics, and anti-platelet agents may also be applied locally to stabilize endothelial cells and reduce lipid content resulting in stabilization of vulnerable plaques.

The drugs which are particularly well suited for the stabilization of vulnerable plaque include, but are not limited to anti-inflammatories including dexamethasone, aspirin, pirfenidone, meclofenamic acid, and tranilast; nonsteroidal anti inflammatories; anti-metabolites, such as 2-chlorodeoxy adenosine (2-CdA or cladribine); immuno-suppressants including sirolimus, everolimus, tacrolimus, etoposide, and mitoxantrone; antithrombins; anti-leukocytes such as 2-CdA, IL-1 inhibitors, anti-CD116/CD118 monoclonal antibodies, monoclonal antibodies to VCAM or ICAM, zinc protoporphyrin; anti-macrophage substances such as drugs that elevate NO, 2-CdA; cyclooxygenase inhibitors including COX-1 and COX-2 inhibitors; cell sensitizers to insulin including glitazones, P par agonists; high density lipoproteins (HDL) and derivatives; and synthetic facsimile of HDL, such as lipator, lovestatin, pranastatin, atorvastatin, simvastatin, and statin derivatives.

Other drugs which may be used to treat inflammation include lipid lowering agents, estrogen and progestin, endothelin receptor agonists and interleukin-6 antagonists, and Adiponectin. Adiponectin inhibits endothelial inflammatory response, suppresses macrophage transformation into foam cells, and inhibits monocyte adhesion to endothelial cells.

Agents for the treatment of ischemic injury may also be delivered using a gene therapy-based approach in combination with an expandable medical device. Gene therapy refers to the delivery of exogenous genes to a cell or tissue, thereby causing target cells to express the exogenous gene product. Genes are typically delivered by either mechanical or vector-mediated methods. Mechanical methods include, but are not limited to, direct DNA microinjection, ballistic DNA-particle delivery, liposome-mediated transfection, and receptor-mediated gene transfer. Vector-mediated delivery typically involves recombinant virus genomes, including but not limited to those of retroviruses, adenoviruses, adeno-associated viruses, herpesviruses, vaccinia viruses, picomaviruses, alphaviruses, and papovaviruses. Gene therapy may be used to inhibit tissue factor by overexpressing tissue factor pathway inhibitor (TFPI) or to promote overexpression of vascular prostacyclin.

According to one aspect of the invention, a stent or other local delivery device is used for local delivery of 2-CdA and/or HDL to the site of a vulnerable plaque and/or to the blood stream upstream of a vulnerable plaque.

In one example, the vulnerable plaque can be located by thermal sensors, magnetic resonance imaging (MRI), elastography, optical coherence tomography (OCT), contrast agents, near-infrared and infrared light techniques, or accumulation of radiopharmaceutical agents. The stent can then be located to deliver the plaque stabilizing agent directly to the vessel wall at the site of the vulnerable plaque. Additionally, stabilizing agent may be delivered luminally into the blood steam for treatment of downstream vulnerable plaques which have or have not been identified. In the case where the location of a vulnerable plaque has not specifically identified, the stent may be placed after a conventional angioplasty procedure and the drug may be delivered primarily to the blood stream to treat potential downstream vulnerable plaque.

The drug can be delivered by a stent containing drug in openings in the stent as described further below. The drug can also be delivered by a drug coated stent, an implant, microspheres, a catheter, coils, or other local delivery means.

The drug can be released over an administration period which is dependent on the mode of action of the drug delivered. For example, HDL may be delivered over an administration period of from hours to months. In another example, a fast acting drug, such as 2-CdA may be delivered over a shorter administration period of a few seconds to a several days, preferably about one to four days.

In one example, the drug for vulnerable plaque stabilization is delivered from a stent primarily in a mural direction with minimal drug being delivered from the stent directly into the blood stream. This allows the drug to be delivered directly to the plaque to be treated with minimal loss of the drug or delivery of the drug to other parts of the body.

In another example, the drug for vulnerable plaque stabilization is delivered from a stent primarily in a luminal direction to treat vulnerable plaque at and downstream of an implantation site.

In an additional example, the drug for vulnerable plaque stabilization is delivered from a stent in both a luminal and mural direction to treat vulnerable plaque at and downstream of an implantation site.

The present invention is also particularly well suited for the delivery of one or more additional therapeutic agents from a mural or luminal side of a stent in addition to the first agent delivered for stabilization of vulnerable plaque. Some examples of other murally delivered agents may include antineoplastics, antiangiogenics, angiogenic factors, antirestenotics, anti-thrombotics, such as heparin, antiproliferatives, such as paclitaxel and Rapamycin and derivatives thereof.

In one dual agent example, a drug suited for the stabilization of vulnerable plaque is delivered primarily luminally from a stent while a drug for the treatment of restenosis is also delivered primarily murally from the stent.

In another dual agent delivery example, two agents for treatment vulnerable plaque are both delivered primarily luminally. The two agents may be delivered over different administration periods depending on the mode of action of the agents. For example, a fast acting agent may be delivered over a short period of a few minutes while a slower acting agent is delivered over several hours or days.

Some of the therapeutic agents for use with the present invention which may be transmitted primarily luminally, primarily murally, or both include, but are not limited to, antiproliferatives including paclitaxel and rapamyacin, antithrombins, immunosuppressants including sirolimus, antilipid agents, anti-inflammatory agents, antineoplastics, antiplatelets, angiogenic agents, anti-angiogenic agents, vitamins, antimitotics, metalloproteinase inhibitors, NO donors, estradiols, anti-sclerosing agents, and vasoactive agents, endothelial growth factors, estrogen, beta blockers, AZ blockers, hormones, statins, insulin growth factors, antioxidants, membrane stabilizing agents, calcium antagonists, retenoid, bivalirudin, phenoxodiol, etoposide, ticlopidine, dipyridamole, and trapidil alone or in combinations with any therapeutic agent mentioned herein. Therapeutic agents also include peptides, lipoproteins, polypeptides, polynucleotides encoding polypeptides, lipids, protein-drugs, protein conjugate drugs, enzymes, oligonucleotides and their derivatives, ribozymes, other genetic material, cells, antisense, oligonucleotides, monoclonal antibodies, platelets, prions, viruses, bacteria, and eukaryotic cells such as endothelial cells, stem cells, ACE inhibitors, monocyte/macrophages or vascular smooth muscle cells to name but a few examples. The therapeutic agent may also be a pro-drug, which metabolizes into the desired drug when administered to a host. In addition, therapeutic agents may be pre-formulated as microcapsules, microspheres, microbubbles, liposomes, niosomes, emulsions, dispersions or the like before they are incorporated into the therapeutic layer. Therapeutic agents may also be radioactive isotopes or agents activated by some other form of energy such as light or ultrasonic energy, or by other circulating molecules that can be systemically administered. Therapeutic agents may perform multiple functions including modulating angiogenesis, restenosis, cell proliferation, thrombosis, platelet aggregation, clotting, and vasodilation. Anti-inflammatories include non-steroidal anti-inflammatories (NSAID), such as aryl acetic acid derivatives, e.g., Diclofenac; aryl propionic acid derivatives, e.g., Naproxen; and salicylic acid derivatives, e.g., aspirin, Diflunisal. Anti-inflammatories also include glucocoriticoids (steroids) such as dexamethasone, prednisolone, and triamcinolone. Anti-inflammatories may be used in combination with antiproliferatives to mitigate the reaction of the tissue to the antiproliferative.

Some of the agents described herein may be combined with additives which preserve their activity. For example additives including surfactants, antacids, antioxidants, and detergents may be used to minimize denaturation and aggregation of a protein drug. Anionic, cationic, or nonionic detergents may be used. Examples of nonionic additives include but are not limited to sugars including sorbitol, sucrose, trehalose; dextrans including dextran, carboxy methyl (CM) dextran, diethylamino ethyl (DEAE) dextran; sugar derivatives including D-glucosaminic acid, and D-glucose diethyl mercaptal; synthetic polyethers including polyethylene glycol (PEO) and polyvinyl pyrrolidone (PVP); carboxylic acids including D-lactic acid, glycolic acid, and propionic acid; detergents with affinity for hydrophobic interfaces including n-dodecyl-β-D-maltoside, n-octyl-β-D-glucoside, PEO-fatty acid esters (e.g. stearate (myrj 59) or oleate), PEO-sorbitan-fatty acid esters (e.g. Tween 80, PEO-20 sorbitan monooleate), sorbitan-fatty acid esters (e.g. SPAN 60, sorbitan monostearate), PEO-glyceryl-fatty acid esters; glyceryl fatty acid esters (e.g. glyceryl monostearate), PEO-hydrocarbon-ethers (e.g. PEO-10 oleyl ether; triton X-100; and Lubrol. Examples of ionic detergents include but are not limited to fatty acid salts including calcium stearate, magnesium stearate, and zinc stearate; phospholipids including lecithin and phosphatidyl choline; CM-PEG; cholic acid; sodium dodecyl sulfate (SDS); docusate (AOT); and taumocholic acid.

Implantable Medical Devices with Openings

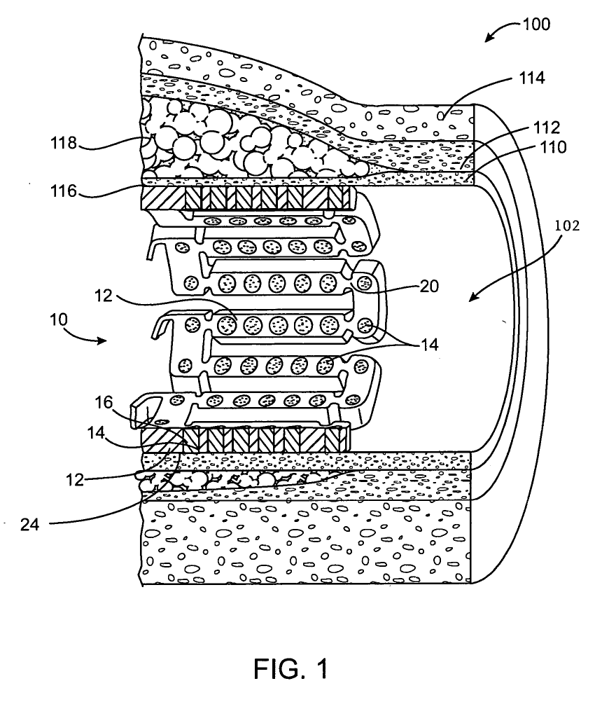

FIG. 1 illustrates an expandable medical device 10 in the form of a stent implanted in a lumen 102 of an artery 100. A wall of the artery 100 includes three distinct tissue layers, the intima 110, the media 112, and the adventitia 114. At the site of a vulnerable plaque, a thin fibrous cap 116 covers a lipid core 118.

When the expandable medical device 10 is implanted in an artery at a vulnerable plaque site, a therapeutic agent delivered from the expandable medical device to the wall of the artery 100 is distributed locally to the tissue at the site of the vulnerable plaque. The therapeutic agent delivered from the expandable medical device to the lumen of the artery 100 treats both the adjacent vulnerable plaque and vulnerable plaque located downstream of the device 10. Preferably, the device 10 is implanted to cover the length of the vulnerable plaque with the stent extending slightly beyond the plaque to ensure stabilization of the entire vulnerable plaque site.

One example of an expandable medical device 10, as shown in FIGS. 1-3, includes large, non-deforming struts 12, which can contain openings 14 without compromising the mechanical properties of the struts, or the device as a whole. The non-deforming struts 12 may be achieved by the use of ductile hinges 20 which are described in detail in U.S. Pat. No. 6,241,762, which is incorporated herein by reference in its entirety. The openings 14 serve as large, protected reservoirs for delivering various beneficial agents to the device implantation site and downstream.

The relatively large, protected openings 14, as described above, make the expandable medical device of the present invention particularly suitable for delivering large amounts of therapeutic agents, larger molecules or genetic or cellular agents, combinations of multiple agents, and for directional delivery of agents. The large non-deforming openings 14 in the expandable device 10 form protected areas or receptors to facilitate the loading of such an agent, and to protect the agent from abrasion, extrusion, or other degradation during delivery and implantation.

FIG. 1 illustrates an expandable medical device for delivery of a therapeutic agent 16. The openings 14 contain the therapeutic agent 16 for delivery both to the wall of the blood vessel and to the lumen of the blood vessel.

The volume of beneficial agent that can be delivered using openings 14 is about 3 to 10 times greater than the volume of a 5 micron coating covering a stent with the same stent/vessel wall coverage ratio. This much larger beneficial agent capacity provides several advantages. The larger capacity can be used to deliver multi-drug combinations, each with independent release profiles, for improved efficacy. Also, larger capacity can be used to provide larger quantities of less aggressive drugs and to achieve clinical efficacy without the undesirable side-effects of more potent drugs, such as retarded healing of the endothelial layer.

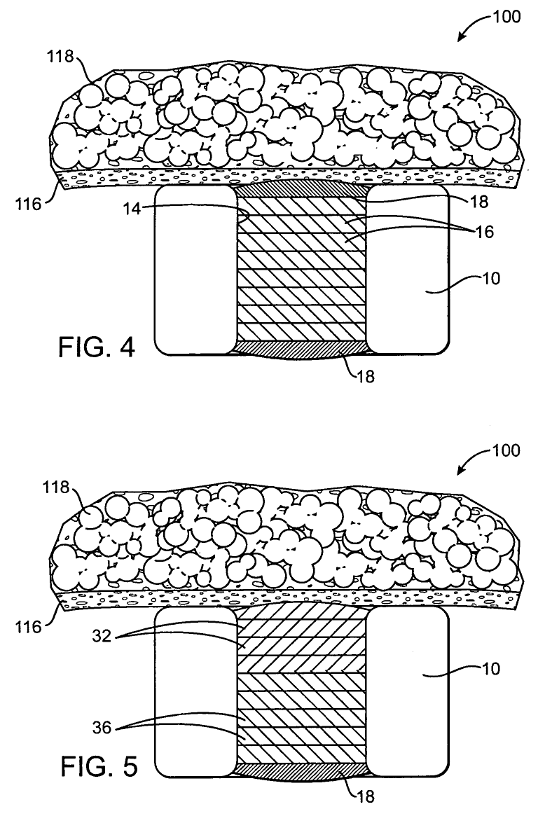

FIG. 4 shows a cross section of a portion of a medical device 10 in which one or more beneficial agents have been loaded into an opening 14 in multiple layers. Although multiple discrete layers are shown for ease of illustration, the layers may be discrete layers with independent compositions or blended to form a continuous polymer matrix and agent inlay. For example, the layers can be deposited separately in layers of a drug, polymer, solvent composition which are then blended together in the openings by the action of the solvent. The agent may be distributed within an inlay uniformly or in a concentration gradient. Examples of some methods of creating such layers and arrangements of layers are described in U.S. Patent Publication No. 2002/0082680, published on Jun. 27, 2002, which is incorporated herein by reference in its entirety. The use of drugs in combination with polymers within the openings 14 allows the medical device 10 to be designed with drug release kinetics tailored to the specific drug delivery profile desired.

According to one example, the total depth of the opening 14 is about 50 to about 140 microns, and the typical layer thickness would be about 2 to about 50 microns, preferably about 12 microns. Each typical layer is thus individually about twice as thick as the typical coating applied to surface-coated stents. There can be at least two and preferably about six to twelve such layers in a typical opening, with a total beneficial agent thickness about 4 to 28 times greater than a typical surface coating. According to one embodiment of the present invention, the openings have an area of at least 5×10⁻⁶ square inches, and preferably at least 10×10⁻⁶ square inches.

In the example of FIG. 4, the luminal and mural sides of the openings 14 are provided with optional barrier/cap layers 18 which are layers of polymer or other material which protect the drug layers or provide for directional delivery. A barrier layer may have an erosion rate which is sufficiently slow to allow substantially all of the therapeutic agent in the therapeutic agent layers 16 to be delivered from the mural or luminal side of the opening, as desired, prior to complete erosion of the barrier layer. The barrier/cap layer 18 on the luminal side of the opening 14 also can provide a seal during filling of the openings. A barrier/cap layer 18 on the mural side can be a rapidly degrading material providing protection during transport, storage or delivery of the stent to the implantation site. The barrier layers 18 may be omitted where mural and luminal delivery of the agent is desired and protection is not needed.

Since each layer of both the barrier 18 and therapeutic agent 16 is created independently, individual chemical compositions and pharmacokinetic properties can be imparted to each layer. Numerous useful arrangements of such layers can be formed, some of which will be described below. Each of the layers may include one or more agents in the same or different proportions from layer to layer. Changes in the agent concentration between layers can be used to achieve a desired delivery profile. For example, a decreasing release of drug for about 24 hours can be achieved. In another example, an initial burst followed by a constant release for about one week can be achieved. Other examples can deliver an agent over a sustained period of time, such as several days to several months. Substantially constant release rates over time period from a few hours to months can be achieved. The layers may be solid, porous, or filled with other drugs or excipients.

FIG. 5 is a cross sectional view of a portion of an expandable medical device 10 including two or more therapeutic agents. Dual agent delivery systems such as that shown in FIG. 5 can deliver two or more therapeutic agents in the same direction or in different directions for the treatment of different conditions or stages of conditions. For example, a dual agent delivery system may deliver one agent primarily in the luminal direction for treatment of vulnerable plaque and another agent primarily in the mural direction for treatment of restenosis from the same drug delivery device opening. Alternately, different drugs may be delivered from different openings.

In FIG. 5, a first agent 36 provided for treating vulnerable plaque is located at the luminal side of the device 10 in one or more layers adjacent a fast degrading cap layer 18. A second therapeutic agent 32 for reducing restenosis is provided at the mural side of the opening in one or more layers. A separating layer (not shown) can be provided between the agent layers to insure complete delivery of each agent to the respective side of the device. A separating layer can be omitted when some delivery in each direction is desired or acceptable.

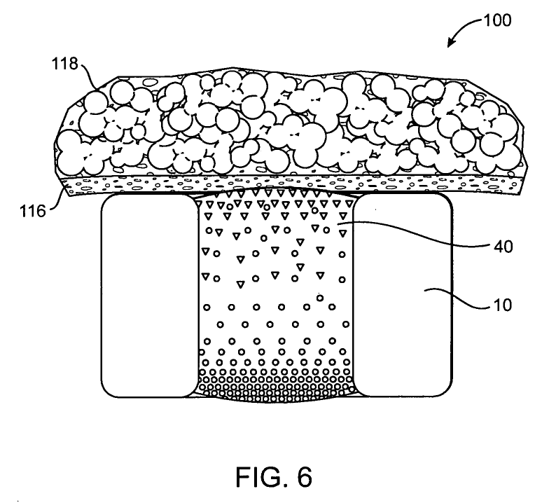

FIG. 6 illustrates an expandable medical device 10 including an inlay 40 formed of a biocompatible matrix with first and second agents provided in the matrix for delivery according to different agent delivery profiles. As shown in FIG. 6, a first drug illustrated by Os is provided in the matrix with a concentration gradient such that the concentration of the drug is highest adjacent the luminal side of the opening and is lowest at the mural side of the opening. The second drug illustrated by As is relatively concentrated in an area close to the mural side of the opening. This configuration illustrated in FIG. 6 results in delivery of two different agents with different delivery profiles or primarily in different directions from the same inlay 40. The two different agents can be agents which treat vulnerable plaque by different modes of action, such as an anti-metabolite agent and an anti-inflammatory agent.

In the embodiments described above, the therapeutic agent can be provided in the expandable medical device in a biocompatible matrix. The matrix can be bioerodible as those described below or can be a permanent part of the device from which the therapeutic agent diffuses. One or more barrier layers, separating layers, and cap layers of the same or different biocompatible matrices can be used to separate therapeutic agents within the openings or to prevent the therapeutic agents from degradation or delivery prior to implantation of the medical device.

### EXAMPLES

### Example 1

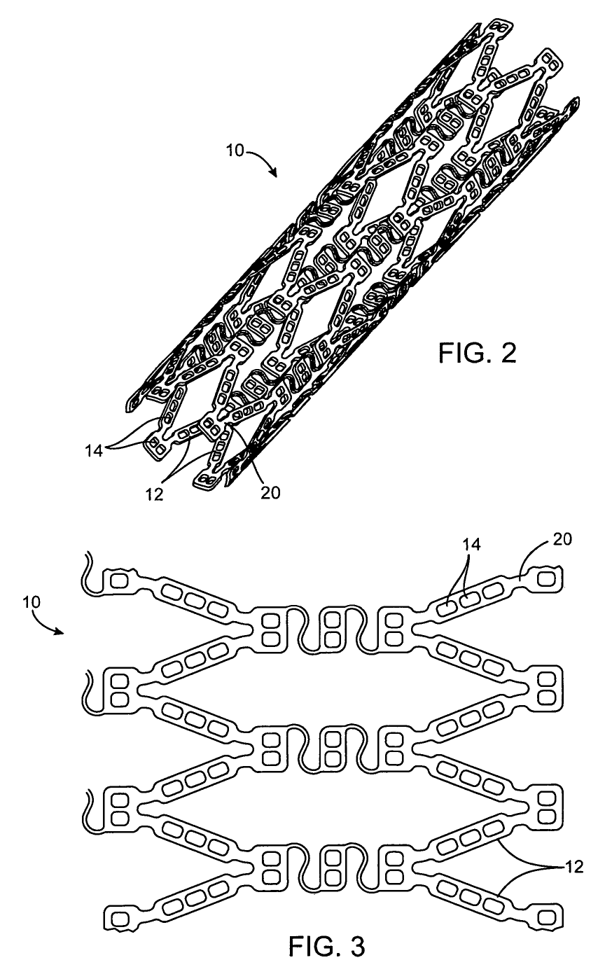

In this example, a drug delivery stent substantially equivalent to the stent illustrated in FIGS. 2 and 3 having an expanded size of about 3 mm×17 mm is loaded with 2-CdA (cladribine) in the following manner. The stent is positioned on a mandrel and a fast degrading barrier layer is deposited into the openings in the stent. The barrier layer is low molecular weight PLGA provided on the luminal side to seal the luminal side of the stent opening during filling. The layers described herein are deposited in a dropwise manner and are delivered in liquid form by use of a suitable organic solvent, such as DMSO, NMP, or DMAc. A plurality of layers of 2-CdA and low molecular weight PLGA matrix are then deposited into the openings to form an inlay of drug for the reduction of ischemic injury. The 2-CdA and polymer matrix are combined and deposited in a manner to achieve a drug delivery profile which results in about 70% release in the first day and the remainder of the drug released in four days. A cap layer of low molecular weight PLGA, a fast degrading polymer, is deposited over the active agent layers protect the active agent during storage, transport, and delivery to the implantation site. The degradation rate of the cap layer is selected so that the agent is delivered relatively quickly after implantation. The total dosage on the stent is about 10 to about 600 micrograms, preferably about 200 to about 400 micrograms, and more preferably about 300 micrograms.

1. A method for treating vulnerable plaque within a blood vessel comprising:
identifying an implantation site in a blood vessel with vulnerable plaque, wherein the implantation site is at or upstream of the vulnerable plaque;
delivering an expandable medical device containing a therapeutic agent which stabilizes the vulnerable plaque to the blood vessel at the selected implantation site;
implanting the medical device at the implantation site; and
delivering the therapeutic agent from the expandable medical device to vessel wall tissue over an administration period sufficient to stabilize the vulnerable plaque.

2. The method of claim 1, wherein the therapeutic agent is an anti-inflammatory.

3. The method of claim 1, wherein the therapeutic agent is a nonsteroidal anti inflammatory.

4. The method of claim 1, wherein the therapeutic agent is an anti-metabolite.

5. The method of claim 1, wherein the therapeutic agent is an immuno-suppressant.

6. The method of claim 1, wherein the therapeutic agent is an antithrombin.

7. The method of claim 1, wherein the therapeutic agent is an anti-leukocyte.

8. The method of claim 1, wherein the therapeutic agent is a high density lipoprotein.

9. The method of claim 1, wherein the therapeutic agent is a cyclooxygenase inhibitor.

10. The method of claim 1, wherein the therapeutic agent is a glitazones or P par agonist.

11. The method of claim 1, wherein the therapeutic agent is contained in a plurality of openings in the device.

12. The method of claim 11, wherein the openings also contain a therapeutic agent for treatment of restenosis.

13. The method of claim 11, wherein the therapeutic agent is arranged in the openings for directional delivery primarily to a luminal side of the device.

14. The method of claim 13, wherein the openings also contain a therapeutic agent for treatment of restenosis arranged for directional delivery primarily to a mural side of the device.

15. An expandable medical device for delivering a therapeutic agent locally to a vulnerable plaque, the device comprising:
an implantable medical device body configured to be implanted within a coronary artery; and
a therapeutic dosage of a therapeutic agent for stabilization of vulnerable plaque, the therapeutic agent affixed in openings in the implantable medical device body in a manner such that the therapeutic agent is released to the vulnerable plaque at a therapeutic dosage and over an administration period effective to stabilize the vulnerable plaque.

16. The device of claim 15, wherein the therapeutic agent is an anti-inflammatory.

17. The device of claim 15, wherein the therapeutic agent is a nonsteroidal anti-inflammatory.

18. The device of claim 15, wherein the therapeutic agent is an anti-metabolite.

19. The device of claim 15, wherein the therapeutic agent is an immuno-suppressant.

20. The device of claim 15, wherein the therapeutic agent is an antithrombin.

21. The device of claim 15, wherein the therapeutic agent is an anti-leukocyte.

22. The device of claim 15, wherein the therapeutic agent is a high density lipoprotein.

23. The device of claim 15, wherein the therapeutic agent is a cyclooxygenase inhibitor.

24. The device of claim 15, wherein the therapeutic agent is a glitazones or P par agonist.

25. The device of claim 15, wherein the therapeutic agent is affixed in the medical device for delivery primarily from a luminal side of the medical device, and further comprising an antiresenotic agent affixed to the medical device for delivery primarily from a mural side of the medical device.

26. The device of claim 15, wherein the therapeutic agent is affixed in the implantable medical device with a biocompatible polymer.
